(19) United States
(12) Patent Application Publication
(10) Pub. No.: US 2024/0003865 A1
(43) Pub. Date: Jan. 4, 2024

(54) ELECTRODE LAMINATE ARRAY, CURVED ELECTRODE ARRAY, METHOD FOR PRODUCING CURVED ELECTRODE ARRAY AND EXTRACELLULAR POTENTIAL MEASUREMENT METHOD

(71) Applicant: NIPPON TELEGRAPH AND TELEPHONE CORPORATION, Toyo (JP)

(72) Inventors: Koji SAKAI, Musashino-shi, Tokyo (JP); Masumi YAMAGUCHI, Musashino-shi, Tokyo (JP); Tetsuhiko TESHIMA, Musashino-shi, Tokyo (JP)

(73) Assignee: NIPPON TELEGRAPH AND TELEPHONE CORPORATION, Tokyo (JP)

(21) Appl. No.: 18/039,462

(22) PCT Filed: Dec. 2, 2020
(86) PCT No.: PCT/JP2020/044849

§ 371 Date: May 30, 2023

(51) Int. Cl. G01N 33/483

(52) U.S. Cl. CPC G01N 33/4836

(57) ABSTRACT

An aspect of the present invention is an electrode stack array including: a planar substrate; and a plurality of electrode stacks, each electrode stack including a sacrificial layer that is stacked on the planar substrate, a bent layer that is stacked on the sacrificial layer and is capable of bending such that a surface on a side opposite to a surface on a side of the sacrificial layer becomes an inner side, and a conductive layer that is disposed on the bent layer.

### TECHNICAL FIELD

The present invention relates to a technology of an electrode stack array, a curved electrode array, a method for manufacturing a curved electrode array, and a method for measuring an extracellular potential.

### BACKGROUND ART

In drug discovery and cell biology, electrical properties are one of important indices for evaluating the functions of cells and organs. In particular, cells of the nervous system and heart transmit information using action potentials, and produce a function as an organ as a result of information transmission of a cell population.

There is a microelectrode array measurement method as a method for measuring electrical activity of cells forming a population at a high throughput from a plurality of points over a long period of time. The most standard method for evaluating the electrical properties of cells is called a patch clamp method in which an electrode is directly inserted into a cell, but this method has a limitation that it is difficult to simultaneously measure a plurality of cells and the cell is damaged. On the other hand, in the microelectrode array measurement method, since a potential change generated outside cells is measured by culturing the cell on a substrate on which a plurality of electrodes is arranged, it is possible to measure a plurality of cells for a long period of time. In addition, since a large number of samples can be obtained at the same time by dividing the sample for each electrode, excellent high throughput is provided. From the above feature, the microelectrode array measurement method is highly expected as a tool for toxicity screening of drugs in the field of drug discovery. In addition, the microelectrode array measurement method is also used as a tool for obtaining biological knowledge such as a spatiotemporal activity pattern as a population of cells and long-term growth of cells.

Moreover, methods for patterning cells on a substrate have been studied so far in order to more effectively take advantage of the features of measurement using a microelectrode array (Non Patent Literature 1 and Non Patent Literature 2). The spatial disposition of cells is controlled by separately forming adherent and non-adherent regions of cells on the substrate by chemical modification or material selection. In addition, there is also a method of structurally patterning by installing wells on a substrate. For example, it is possible to enhance throughput by disposing cells only immediately above electrodes and separating samples for each electrode and dispose cells in a lattice pattern on the electrode substrate so as to evaluate the relationship between the shape of the network and spatiotemporal information transmission.

### CITATION LIST

### Non Patent Literature

Non Patent Literature 1: A. Natarajan et al., Patterned Cardiomyocytes on Microelectrode Arrays as a Functional, High Information Content Drug Screening Platform. Biomaterials, 32, 18 (2011): 4267-4274 Non Patent Literature 2: S. B. Jun et al., Low-Density Neuronal Networks Cultured using Patterned Poly-L-Lysine on Microelectrode Arrays. Journal of neuroscience methods, 160, 2 (2007): 317-326

### SUMMARY OF INVENTION

### Technical Problem

However, while the microelectrode array measurement method has the above-described advantages, there is a problem that the amplitude of the measured extracellular potential is small in the case of using cell patterning. The small amplitude of the extracellular potential is due to the small number of cells. That is, in the microelectrode array measurement method, the total number of cells engrafted on the substrate is small because the adhesion region is limited as compared with the case of uniform culture without patterning. Therefore, the amount of growth factors released by the cells themselves is small, and the proliferation and growth of the cells are slow. As a result, since the covering of the electrode by the cells is reduced, the current generated around the cells does not flow through the electrode but diffuses into the culture solution, and the extracellular potential decreases.

In view of the above circumstances, an object of the present invention is to provide a technology capable of measuring an extracellular potential at a higher S/N using the microelectrode array measurement method.

### Solution to Problem

An aspect of the present invention is an electrode stack array including: a planar substrate; and a plurality of electrode stacks, each electrode stack including a sacrificial layer that is stacked on the planar substrate, a bent layer that is stacked on the sacrificial layer and is capable of bending such that a surface on a side opposite to a surface on a side of the sacrificial layer becomes an inner side, and a conductive layer that is disposed on the bent layer.

An aspect of the present invention is a curved electrode array including: a planar substrate; and a plurality of curved electrodes, each curved electrode including a bent layer that is curved to form an internal space, and a conductive layer that is disposed on an inner surface of the bent layer.

An aspect of the present invention is a method for manufacturing a curved electrode array including: a step of preparing the electrode stack array; and a step of removing the sacrificial layer of the electrode stack array and detaching the planar substrate and the bent layer so as to curve the bent layer such that a surface on a side opposite to a surface on a side of the sacrificial layer becomes an inner side.

An aspect of the present invention is a method for measuring an extracellular potential, including: a step of preparing the electrode stack array; a step of attaching a cell to a surface of the conductive layer of the electrode stack array; a step of forming a curved electrode array by removing the sacrificial layer of the electrode stack array and detaching the planar substrate and the bent layer so as to curve the bent layer such that a surface on a side opposite to a surface on a side of the sacrificial layer becomes an inner side; and a step of measuring an extracellular potential of the cell attached to the conductive layer of a curved electrode of the curved electrode array.

### Advantageous Effects of Invention

According to the present invention, it is possible to measure an extracellular potential at a higher S/N using the microelectrode array measurement method.

### BRIEF DESCRIPTION OF DRAWINGS

FIG. 1 is a perspective diagram illustrating an example of an electrode stack array according to an embodiment of the present invention.

FIG. 2 is a plan diagram of the electrode stack array illustrated in FIG. 1.

FIG. 3 is a cross-sectional diagram taken along line of FIG. 2.

FIG. 4 is a diagram illustrating an example of a configuration of an electrode stack included in the electrode stack array illustrated in FIG. 1, FIG. 4(a) is a perspective diagram, and FIG. 4(b) is a cross-sectional diagram taken along line IV-IV of FIG. 4(a).

FIG. 5 is a diagram illustrating an example of a curved electrode including the electrode stack illustrated in FIG. 4, FIG. 5(a) is a perspective diagram, and FIG. 5(b) is a cross-sectional diagram taken along line V-V of FIG. 5(a).

FIG. 6 is a perspective diagram illustrating an example of a state in which cells are encapsulated in the curved electrode illustrated in FIG. 5.

FIG. 7 is a diagram illustrating another example of a configuration of an electrode stack included in the electrode stack array illustrated in FIG. 1, FIG. 7(a) is a perspective diagram, and FIG. 7(b) is a cross-sectional diagram taken along line VII-VII of FIG. 7(a).

FIG. 8 is a diagram illustrating an example of a curved electrode including the electrode stack illustrated in FIG. 7, FIG. 8(a) is a perspective diagram, and FIG. 8(b) is a cross-sectional diagram taken along line VIII-VIII of FIG. 8(a).

FIG. 9 is a plan diagram illustrating another example of an electrode stack array according to an embodiment of the present invention.

FIG. 10 is a cross-sectional diagram taken along line X-X of FIG. 9.

FIG. 11 is a diagram illustrating an example of a configuration of an electrode stack included in the electrode stack array illustrated in FIG. 9, FIG. 11(a) is a perspective diagram, and FIG. 11(b) is a cross-sectional diagram taken along line XI-XI of FIG. 11(a).

FIG. 12 is a diagram illustrating an example of a state in which the electrode illustrated in FIG. 11 is curved, FIG. 12(a) is a perspective diagram, and FIG. 12(b) is a cross-sectional diagram taken along line XII-XII of FIG. 12(a).

FIG. 13 is a perspective diagram illustrating an example of a curved electrode array according to an embodiment of the present invention.

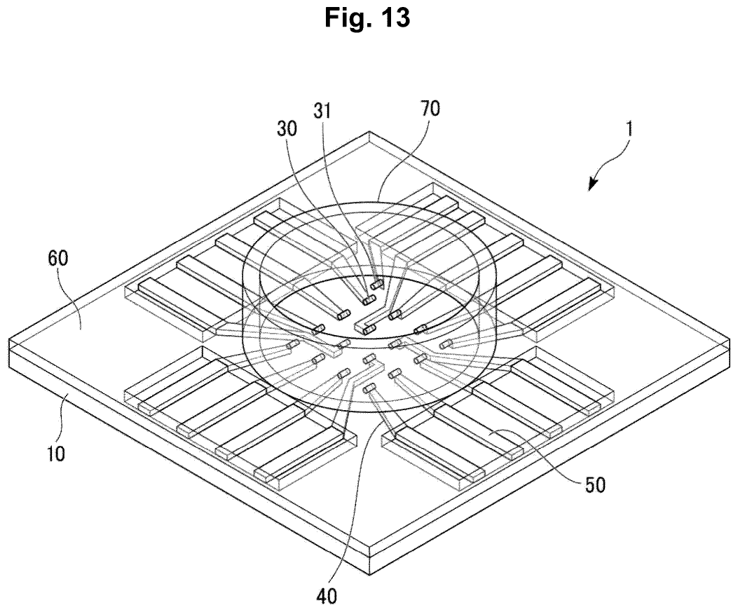

FIG. 14 is a block diagram of a measurement device that can be used for carrying out the method for measuring an extracellular potential according to an embodiment of the present invention.

FIG. 15 is an optical microscope photograph illustrating an electrode portion of an electrode stack array produced in a first example.

FIG. 16 is a differential interference microscope photograph of a curved electrode of the curved electrode array produced in a second example.

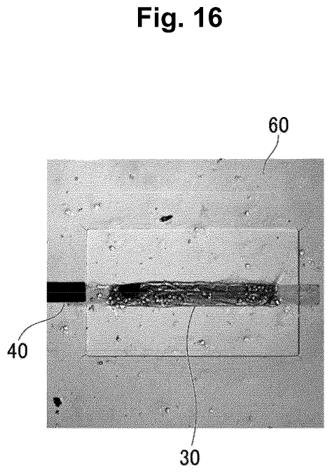

FIG. 17 is a stained image by an Anti-Cardiac Troponin T antibody of the curved electrode of the curved electrode array produced in the second example.

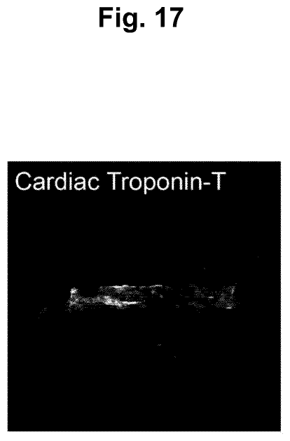

FIG. 18(a) is an activity waveform of a nerve cell on Day 16 cultured in a third example, and FIG. 18(B) is an activity waveform of a nerve cell on Day 16 cultured in a first comparative example.

FIG. 19 is a graph illustrating the number of days of culturing nerve cells and a spike rate measured in the third example and the first comparative example.

### DESCRIPTION OF EMBODIMENTS

Hereinafter, an electrode stack array, a curved electrode array, a method for manufacturing the curved electrode array, and a method for measuring an extracellular potential according to an embodiment of the present invention will be described in detail with reference to the accompanying drawings. Note that, in the drawings, the same or corresponding parts are denoted by the same or corresponding reference numerals, and redundant description will be omitted. Note that the dimensional ratios in the drawings are exaggerated for the sake of description, and do not necessarily coincide with the actual dimensional ratios.

### [Electrode Stack Array]

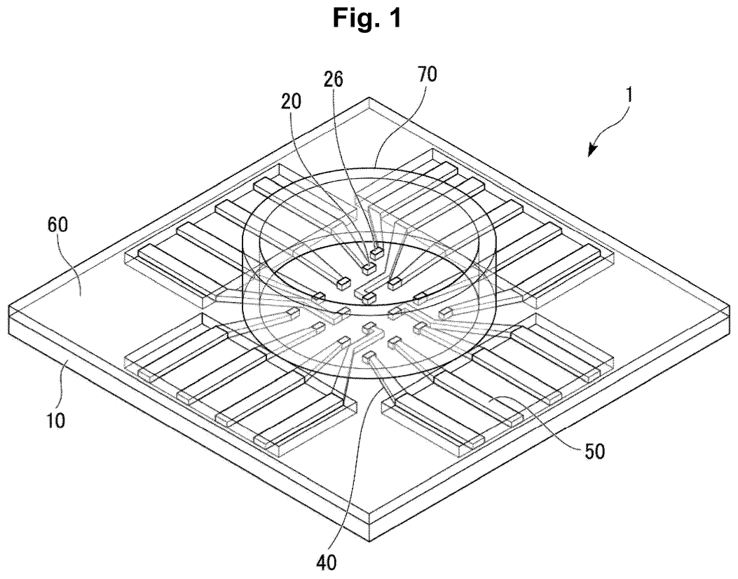

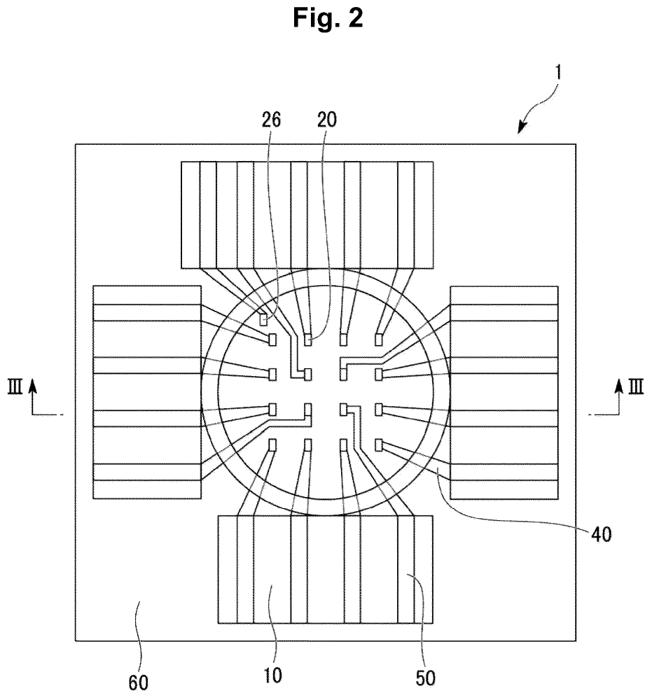

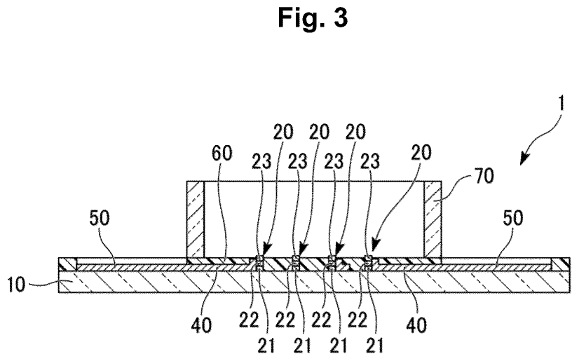

FIG. 1 is a perspective diagram illustrating an example of an electrode stack array according to an embodiment of the present invention. FIG. 2 is a plan diagram of the electrode stack array illustrated in FIG. 1. FIG. 3 is a cross-sectional diagram taken along line III-III of FIG. 2.

An electrode stack array 1 includes a planar substrate 10, a plurality of electrode stacks 20 disposed on one surface of the planar substrate 10, wirings 40 independently connected to each of the electrode stacks 20, connection pads 50 having one end portion connected to the wiring 40 and the other end portion connected to an extracellular potential measurement device (not illustrated), an insulating layer 60 covering those other than the electrode stacks 20 and the connection pads 50, and a culture ring 70 surrounding the plurality of electrode stacks 20. The plurality of electrode stacks 20 is electrodes for culturing cells and measuring the extracellular potential. A reference electrode stack 26 is disposed around the electrode stacks 20, and by measuring a potential difference between each conductive layer of the electrode stacks 20 and a conductive layer of the reference electrode stack 26, a change in extracellular potential of cells cultured in the plurality of electrode stacks 20 can be measured.

### (Electrode Stack and Reference Electrode Stack 26)

Each of the electrode stack 20 and the reference electrode stack 26 is a stack including a sacrificial layer 21 stacked on the planar substrate 10, a bent layer 22 stacked on the sacrificial layer 21, and a conductive layer 23 disposed on the bent layer. The bent layer 22 is capable of bending such that a surface (that is, the surface on the conductive layer 23 side) on a side opposite to a surface on the sacrificial layer 21 side becomes an inner side. That is, the electrode stack 20 is configured such that when the sacrificial layer 21 is removed and the planar substrate 10 and the bent layer 22 are separated, the bent layer 22 is spontaneously curved such that the surface on a side opposite to the surface on the sacrificial layer 21 side becomes an inner side, and a curved electrode having a cylindrical shape is formed. Note that, in the present embodiment, the bent layer 22 is capable of bending in a cylindrical shape. The electrode stack 20 and the reference electrode stack 26 may be an opening type electrode in which the wiring 40 is connected to the opening of the curved electrode when the electrode is curved in a cylindrical shape, or may be a side surface portion type electrode in which the wiring 40 is connected to the side surface portion of the curved electrode when the electrode is curved in a cylindrical shape.

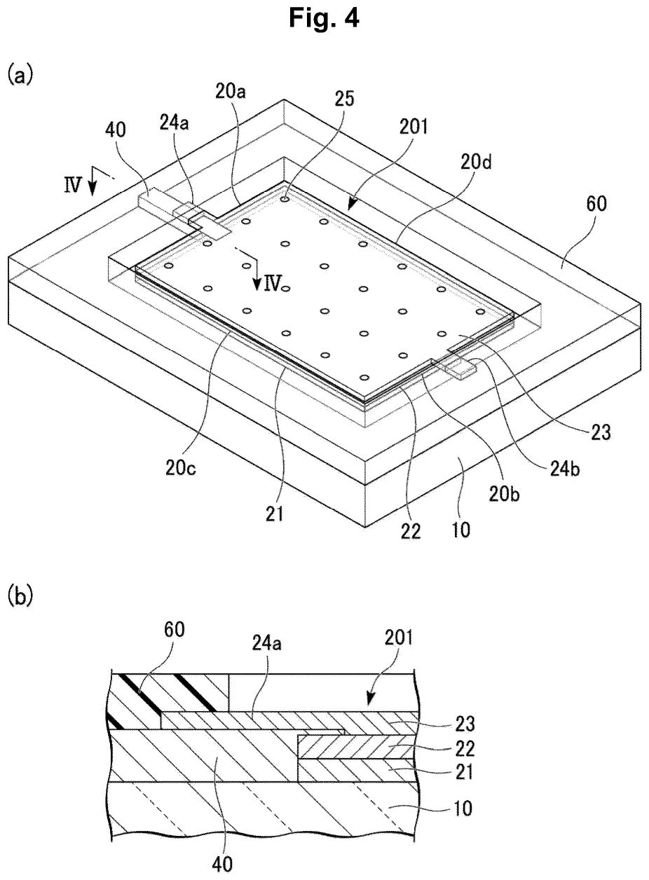

FIG. 4 is a perspective diagram illustrating an example of a configuration of an opening type electrode, FIG. 4(a) is a perspective diagram and FIG. 4(b) is a cross-sectional diagram taken along line IV-IV of FIG. 4(a). As illustrated in FIG. 4, in an opening type electrode stack 201, the conductive layer 23 includes a shaft portion 24a on a side portion 20a connected to the wiring 40 and a shaft portion 24b on a side portion 20b opposite from the side portion 20a. Ends of the shaft portions 24a and 24b are each covered with the insulating layer 60.

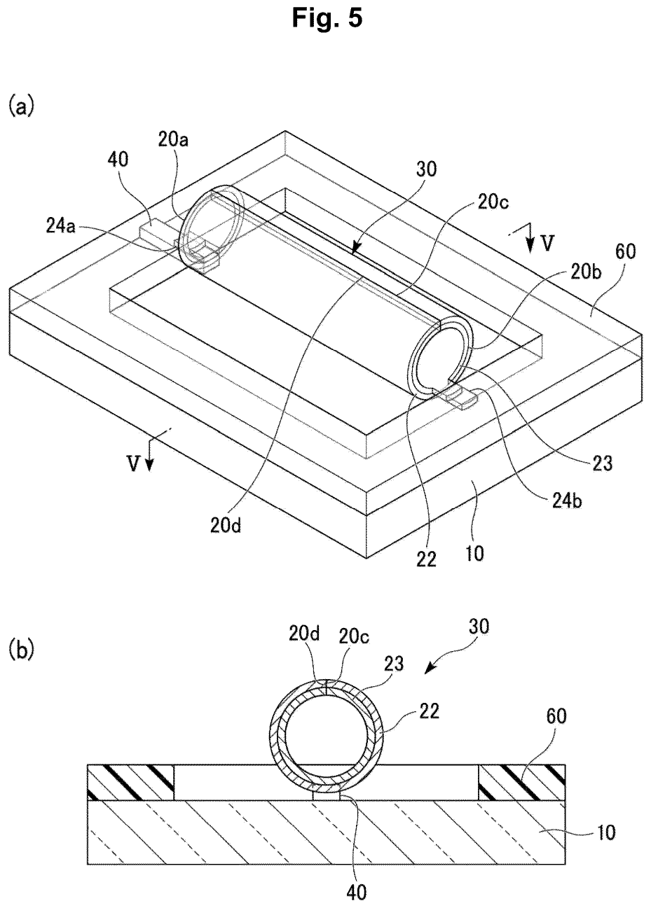

FIG. 5 is a diagram illustrating an example of a state in which the opening type electrode illustrated in FIG. 4 is curved, FIG. 5(a) is a perspective diagram and FIG. 5(b) is a cross-sectional diagram taken along line V-V of FIG. 5(a). When the sacrificial layer 21 of the opening type electrode stack 201 illustrated in FIG. 4 is removed and the planar substrate 10 and a bent layer 22a are separated, as illustrated in FIG. 5, the bent layer 22a is spontaneously curved by using a line connecting the shaft portion 24a and the shaft portion 24b as an axis and forms a curved electrode 30 having a cylindrical shape. The resulting curved electrode 30 having a cylindrical shape is curved such that a left-side side portion 20c and a right-side side portion 20d are in contact with each other when viewed from the wiring 40. The shaft portions 24a and 24b have an action of preventing the curved electrode 30 from greatly separating from the planar substrate 10 and an action of defining the axial direction when the bent layer 22 is curved.

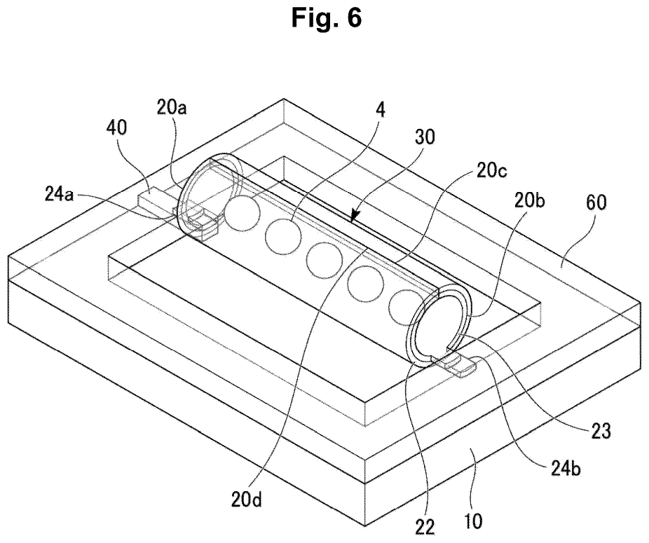

FIG. 6 is a perspective diagram illustrating an example of a state in which cells are encapsulated in the curved electrode illustrated in FIG. 5.

When cells are cultured using the curved electrode 30, cells 4 are seeded on the conductive layer 23 of the opening type electrode stack 201, and then the sacrificial layer 21 is removed to form the curved electrode 30. Thus, as illustrated in FIG. 6, the cells 4 can be present in the internal space of the curved electrode 30.

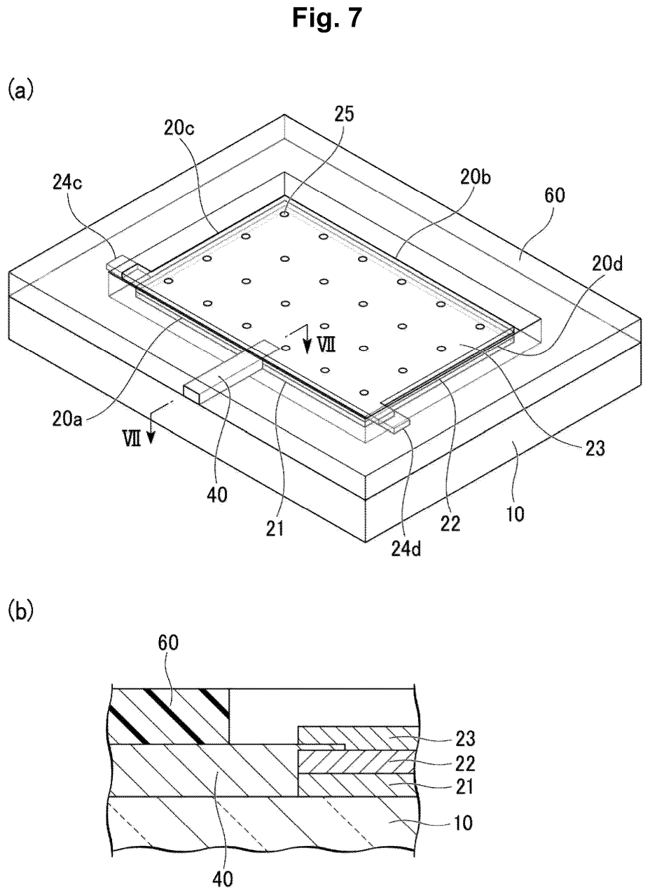

FIG. 7 is a diagram illustrating an example of a configuration of a side surface portion type electrode, FIG. 7(a) is a perspective diagram and FIG. 7(b) is a cross-sectional diagram taken along line VII-VII of FIG. 7(a). As illustrated in FIG. 7, in a side surface portion type electrode stack 202, the conductive layer 23 includes a shaft portion 24c at an end portion of the left-side side portion 20c on the wiring 40 side when viewed from the wiring 40 and a shaft portion 24d at an end portion of the right-side side portion 20d on the wiring 40 side. Ends of the shaft portions 24c and 24d are each covered with the insulating layer 60.

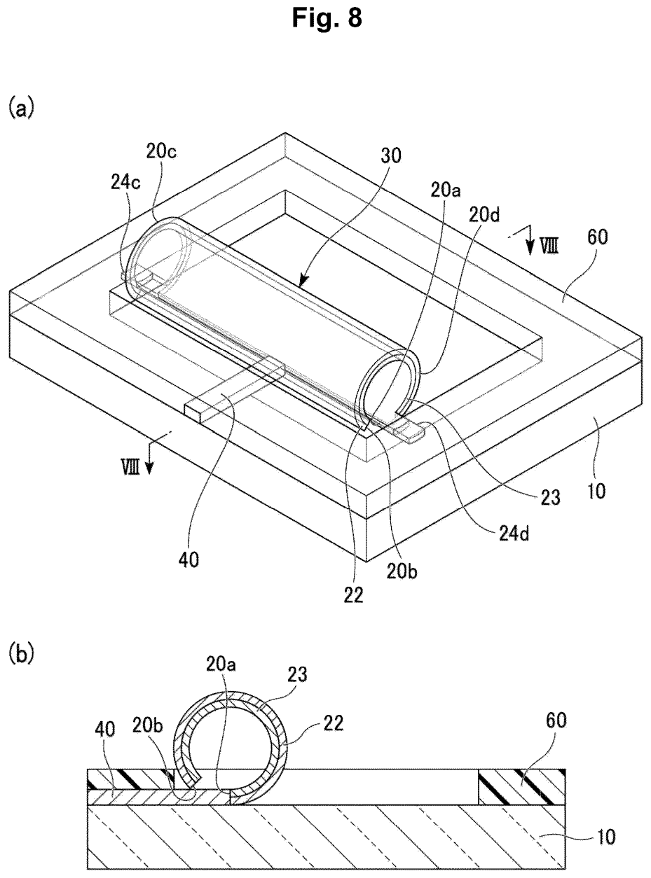

FIG. 8 is a diagram illustrating an example of a curved electrode including the side surface portion type electrode illustrated in FIG. 7, FIG. 8(a) is a perspective diagram, and FIG. 8(b) is a cross-sectional diagram taken along line VIII-VIII of FIG. 8(a). When the sacrificial layer 21 of the side surface portion type electrode stack 202 illustrated in FIG. 7 is removed and the planar substrate 10 and a bent layer 22b are separated, as illustrated in FIG. 8, the bent layer 22b is spontaneously curved by using a line connecting the shaft portion 24a and the shaft portion 24b as an axis and forms a curved electrode 30 having a cylindrical shape. The resulting curved electrode 30 having a cylindrical shape is curved such that the side portion 20a connected to the wiring 40 and the side portion 20b opposite to the side portion 20a are in contact with each other.

### (Sacrificial Layer 21)

The sacrificial layer 21 serves as an adhesive layer for fixing the planar substrate 10 and the bent layer 22. As the material of the sacrificial layer 21, for example, a material having a property of dissolving in response to an external stimulus such as a chemical substance, a temperature change, or light irradiation within a range not affecting cell engraftment, or a biodegradable material can be used. More specifically, a water-soluble inorganic material, a water-soluble polymer material, and calcium alginate gel can be used. Examples of the water-soluble inorganic material include silicon oxide, magnesium, and germanium. Examples of the water-soluble polymer material include polycaprolactone, polylactic acid, polyvinyl alcohol, and gelatin. The calcium alginate gel can be dissolved by an external stimulus within a range not affecting the cell engraftment. That is, the calcium alginate gel is dissolved by being transferred from the gel to the sol by being brought into contact with a chemical substance such as a chelating agent such as sodium citrate or ethylenediaminetetraacetic acid (EDTA) or an enzyme called alginate lyase. Since the chelating agent and the enzyme are not toxic to cells, it is possible to encapsulate the cells 4 in the curved electrode 30 by seeding the target cells 4 on the conductive layer 23 immediately before dissolving the sacrificial layer 21 using the calcium alginate gel.

The thickness of the sacrificial layer 21 is not particularly limited, and may be, for example, within a range of 20 nm or more and 1000 nm or less from the viewpoint of the adhesive force between the planar substrate 10 and the bent layer 22 and the dissolution rate of the sacrificial layer 21.

### (Bent Layer)

The bent layer 22 serves as a substrate that supports the conductive layer 23 of the curved electrode 30. The bent layer 22 has a planar shape when fixed to the planar substrate 10 via the sacrificial layer 21. The material of the bent layer 22 is not limited as long as the bent layer has deformability of being spontaneously curved from a planar state while maintaining adhesion with the conductive layer 23 when the sacrificial layer 21 is removed and the bent layer 22 is separated from the planar substrate 10. The bent layer 22 preferably has optical transparency. Moreover, the bent layer 22 is preferably bioinert. The bent layer 22 may be a single layer having deformability, or may be a stack in which two or more single layers of different materials are stacked so as to have deformability. Examples of the single layer having deformability include a polymer material layer in which a gradient is imparted to the degree of polymerization. The polymer material layer having a gradient in the degree of polymerization can be formed, for example, by changing the amount of light with respect to a photoresist layer such as SU-8. In addition, examples of the combination for the stack having deformability include a combination having different thermal expansion coefficients such as a polymer material layer and a metal material layer and a semiconductor material layer and a metal material layer, a combination of hydrogel layers having different volume change amounts due to swelling, and a combination of a polymer material layer and a graphene layer.

The thickness of the bent layer 22 is not particularly limited, and may be, for example, within a range of 100 nm or more and 10000 nm or less. The bent layer 22 is rectangular, particularly oblong. The bent layer 22 is preferably the same as or larger than the conductive layer 23. The size of the bent layer 22 is not particularly limited, and may be, for example, within a range of 10 μm or more and 1000 μm or less in length and within a range of 10 μm or more and 1000 μm or less in width.

### (Conductive Layer)

The conductive layer 23 is curved together with the bent layer 22 to serve to form the curved electrode 30. The conductive layer 23 preferably has optical transparency. The material of the conductive layer 23 is not limited as long as it is a bioinert and conductive material. As the material of the conductive layer 23, for example, a metal, a conductive oxide, a conductive polymer, or a conductive carbon material can be used. Examples of the metal include gold and platinum. Examples of the conductive oxide include indium tin oxide (ITO). Examples of the conductive polymer include PEDOT (poly (3,4-ethylenedioxythiophene)). Examples of the conductive carbon material include graphene and carbon nanotubes. Moreover, in order to enhance the electrode activity of the conductive layer 23, the surface of the conductive layer 23 may be plated with a conductive material such as platinum black, carbon nanotube, or PEDOT.

The thickness of the conductive layer 23 is not particularly limited within a range not interfering with bending, and may be, for example, within a range of 0.1 nm or more and 100 nm or less. The shape of the conductive layer 23 is not particularly limited, and may be, for example, rectangular, particularly oblong, or may be circular. The size of the conductive layer 23 is not particularly limited, and, in the case of rectangular, from a viewpoint of reducing current leakage, may be, for example, within a range of 10 μm or more and 1000 μm or less in length and within a range of 10 μm or more and 1000 μm or less in width.

### (Planar Substrate)

The planar substrate 10 adheres to the bent layer 22 via the sacrificial layer 21, and serves to hold the bent layer 22 in a planar shape. Therefore, the planar substrate 10 preferably has a flat surface. In addition, the planar substrate 10 preferably has no conductivity. Moreover, when the electrode stack 20 is observed using a transmission microscope, the planar substrate 10 preferably has high transparency and a shape that does not interfere with the operation of the objective lens of the transmission microscope.

As the planar substrate 10, for example, a glass substrate, a polyimide substrate, or a polyethylene terephthalate substrate can be used. Among these substrates, the glass substrate is preferable. The thickness of the planar substrate 10 is not particularly limited, and may be, for example, within a range of 200 μm or more and 1000 μm or less.

### (Wiring and Connection Pad)

The wiring 40 serves to connect the conductive layers 23 of the electrode stack 20 and the reference electrode stack 26 to the connection pads 50. The connection pad 50 serves to connect the wiring 40 and an external measurement device.

The material of each of the wiring 40 and the connection pad 50 is not limited as long as it is a bioinert and highly conductive material. As the material of the wiring 40 and the connection pad 50, for example, a metal, a conductive oxide, a conductive polymer, or a conductive carbon material can be used. Examples of the metal include gold and platinum. Examples of the conductive oxide include indium tin oxide (ITO). Examples of the conductive polymer include PEDOT. Examples of the conductive carbon material include graphene and carbon nanotubes. The material of the wiring 40 and the material of the connection pad 50 may be the same as or different from each other.

The width and thickness of the wiring 40 and the connection pad 50 are not particularly limited. The width of each of the wiring 40 and the connection pad 50 is preferably within a range of 1 μm or more and 100 μm or less. The thickness of each of the wiring 40 and the connection pad 50 is not particularly limited, and may be, for example, within a range of 50 nm or more and 1000 nm or less.

### (Insulating Layer)

The insulating layer 60 serves to prevent the current flowing through the wiring 40 from diffusing to the outside when a cell-containing culture solution is brought into contact with the conductive layer 23 of the electrode stack 20. In addition, the insulating layer 60 serves to fix the shaft portions 24, 24a, and 24b of the conductive layer 23. The material is not limited as long as the insulating layer 60 does not have conductivity. The material of the insulating layer 60 preferably has high cell engraftment. As the material of the insulating layer for example, a photoresist or a polymer material can be used. Examples of the photoresist include OFPR, SU-8, and S1800 series. Examples of the polymer material include poly-para-xylene and polyimide.

The thickness of the insulating layer 60 is not particularly limited, and may be, for example, within a range of 1 μm or more and 10 μm or less.

### (Culture Ring)

The culture ring 70 serves as a container of a culture solution when the cell-containing culture solution and the conductive layer 23 of the electrode stack 20 are brought into contact with each other to attach the cells to the surface of the conductive layer 23 or culture the cells. The material of the culture ring 70 is not limited as long as it is bioinert. As the material of the culture ring 70, for example, silicone rubber or borosilicate glass can be used. The inner diameter of the culture ring 70 is not particularly limited as long as it can surround the conductive layers 23 of the plurality of electrode stacks inside, and may be, for example, within a range of 5 mm or more and 30 mm or less. The height of the culture ring is not particularly limited, and may be within a range of 1 mm or more and 20 mm or less in order to indwell the culture solution.

### [Method for Manufacturing Electrode Stack Array]

The electrode stack array 1 of the present embodiment can be manufactured, for example, by a method including steps (1) to (6) described below.

(1) A step of forming the sacrificial layer 21 on one surface of the planar substrate 10. In this step, the method for forming the sacrificial layer 21 is not particularly limited, and a method generally used for thin film formation can be appropriately selected according to the material of the sacrificial layer 21. Examples of the method for forming the sacrificial layer 21 include a chemical vapor deposition method, a spin coating method, an inkjet printing method, a vapor deposition method, and an electrospray method.

(2) A step of forming the bent layer 22 on the surface of the sacrificial layer 21. In this step, as a method for forming the bent layer 22, a method generally used for thin film formation can be appropriately selected. Examples of the method for forming the bent layer 22 include a chemical vapor deposition method, a spin coating method, an inkjet printing method, a vapor deposition method, a sputtering method, an electrolytic plating method, and an atomic layer deposition method.

(3) A step of forming the wiring 40 and the connection pad 50. In this step, the method for forming the wiring 40 and the connection pad 50 is not particularly limited, and a method generally used for thin film formation can be appropriately selected according to the material of the wiring 40 and the connection pad 50. Examples of the method for forming the wiring 40 and the connection pad 50 include a spin coating method, a vapor deposition method, a sputtering method, an inkjet printing method, a wet etching method, and a lift-off method.

(4) A step of forming the conductive layer 23 on the surface of the bent layer 22. In this step, as a method for forming the conductive layer 23, a method generally used for thin film formation can be appropriately selected. Examples include a chemical vapor deposition method, a spin coating method, an inkjet printing method, a vapor deposition method, a sputtering method, an electrolytic plating method, and an atomic layer deposition method. On the conductive layer 23 formed, the shaft portions 24a to 24d may be formed by etching. Through this step, the electrode stack 20 and the reference electrode stack 26 in which the sacrificial layer 21, the bent layer 22, and the conductive layer 23 are stacked are produced.

(5) A step of forming the insulating layer 60. In this step, the insulating layer 60 is formed so as to cover the surfaces of the planar substrate 10 and the wiring 40 excluding the conductive layer 23 and the connection pads That is, the insulating layer 60 is formed after a mask is disposed on the surfaces of the conductive layers 23 and the connection pads 50. The method for forming the insulating layer 60 is not particularly limited, and a method generally used for thin film formation can be appropriately selected according to the material of the insulating layer 60. Examples of the method for forming the insulating layer 60 include a chemical vapor deposition method and a spin coating method.

(6) A step of disposing the culture ring 70. In this step, the separately prepared culture ring 70 is disposed so as to surround the conductive layers 23 of the plurality of electrode stacks 20 inside, and is fixed with an adhesive. The adhesive is not particularly limited, and for example, a polydimethylsiloxane adhesive may be used.

In the electrode stack array 1 of the present embodiment configured as described above, since the electrode stack 20 including the sacrificial layer 21, the bent layer 22, and the conductive layer 23 is stacked on the planar substrate, it is possible to form the curved electrode 30 by removing the sacrificial layer 21 and separating the planar substrate 10 from the bent layer 22. Then, by encapsulating the cells 4 in the curved electrode 30, diffusion of the growth factors of the cells 4 to the outside is suppressed, so that proliferation and growth of the cells 4 are accelerated. Therefore, the amplitude of the extracellular potential measured using the microelectrode array measurement method increases. In addition, the curved electrode 30 prevents the current flowing through the conductive layer 23 from diffusing to the outside, so that the S/N ratio is improved. For this reason, it is possible to measure a minute amplitude, which is conventionally considered as noise, as the activity of the cells. For the above reasons, by using the electrode stack array 1 of the present embodiment, it is possible to measure the extracellular potential at a higher S/N using the microelectrode array measurement method.

In the electrode stack array 1 of the present embodiment, when the cells 4 are attached to the conductive layer 23, since the conductive layer 23 is planar, the cells 4 are easily attached to the conductive layer 23. In addition, since the disposition of the cells 4 is determined by the structure of the curved electrode 30, the patterning of the cells 4 can be performed simultaneously with the culture of the cells. For example, in the case of the curved electrode 30 having a cylindrical shape, the cells 4 are patterned in a tubular shape in the curved electrode 30 having a cylindrical shape. Moreover, by using the electrode stack array 1 of the present embodiment, it is possible to easily perform high-throughput analysis in which the cells 4 to be attached are changed for each conductive layer 23 and evaluation of the activity pattern with respect to the shape of the cell population.

In the electrode stack array 1 of the present embodiment, when each of the bent layer 22 and the conductive layer 23 is made of a material having optical transparency, the cells 4 encapsulated in the curved electrode 30 can be observed using an optical microscope. This makes it possible to evaluate the relationship between the change in morphological characteristics such as proliferation and growth of the cells 4 and the change in functional characteristics seen from the electrical activity. Note that having optical transparency means that transmittance of visible light (light having a wavelength of 400 to 760 nm) is 90% or more.

In the electrode stack array 1 of the present embodiment, when the conductive layer 23 includes the conductive carbon material, the conductive carbon material is bioinert and does not interfere with the proliferation and growth of the cells 4. Therefore, the cells 4 can be cultured for a long period of time.

In the electrode stack array 1 of the present embodiment, when the curved electrode 30 formed by the curvature of the bent layer 22 has a tubular shape such as a cylinder, the cells 4 can be unfailingly encapsulated in the curved electrode 30, so that the amplitude of the obtained extracellular potential is further increased.

### (Modification)

In the electrode stack array 1 of the present embodiment, the bent layer 22 is oblong and is configured to be curved to form a cylinder, but the shape of the bent layer 22 is not limited thereto. The shape of the bent layer 22 is not particularly limited as long as it can be deformed into a shape that forms an internal space by being curved. For example, the bent layer 22 may be oblong and configured to form a spiral shape by being curved. In addition, the bent layer 22 may have a shape of a developed diagram of a cube and be configured to be curved to form a curved electrode of the cube. Moreover, the bent layer 22 may have a fan shape and be configured to be curved to form a curved electrode having a conical shape without a bottom surface.

In addition, in the electrode stack array 1 of the present embodiment, the number of the conductive layers 23 disposed on the bent layer 22 of the electrode stack 20 is one, but the number of the conductive layers 23 of the electrode stack 20 is not limited thereto. The number of the conductive layers 23 disposed on the bent layer 22 may be two or more.

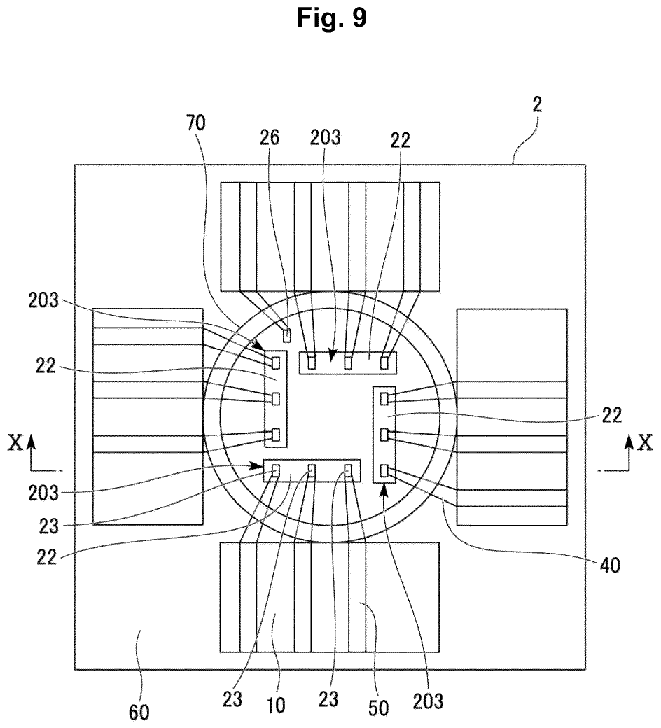

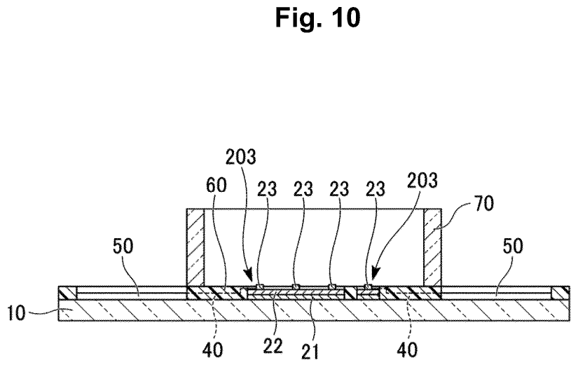

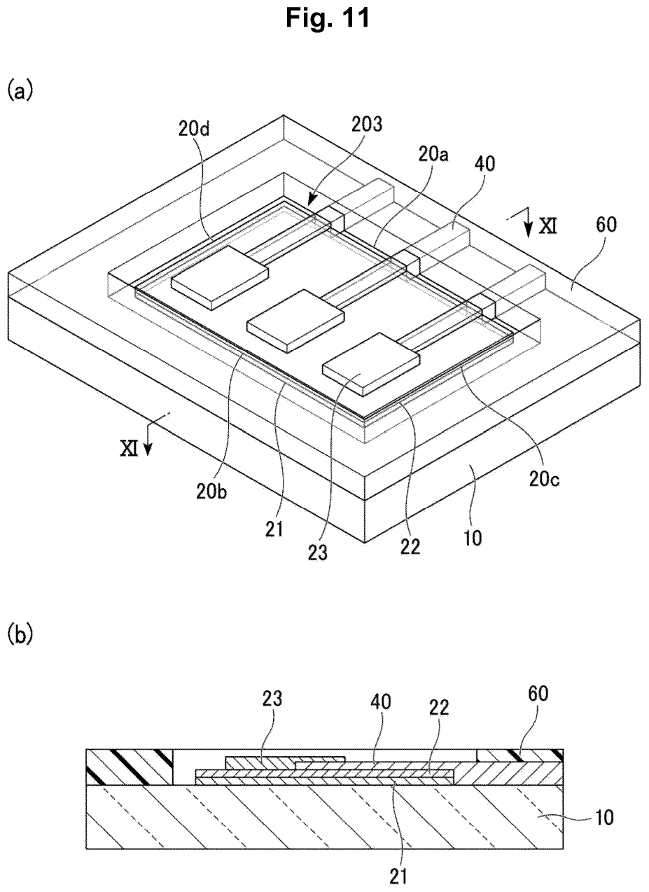

FIG. 9 is a plan diagram illustrating another example of an electrode stack array according to an embodiment of the present invention. FIG. 10 is a cross-sectional diagram taken along line X-X of FIG. 9. FIG. 11 is a diagram illustrating an example of a configuration of an electrode stack included in the electrode stack array illustrated in FIG. 9, FIG. 11(a) is a perspective diagram, and FIG. 11(b) is a cross-sectional diagram taken along line XI-XI of FIG. 11(a).

An electrode stack array 2 illustrated in FIG. 9 has four electrode stacks 203. In each of the four electrode stacks 203, three conductive layers 23 are disposed on the bent layer 22. Each of the three conductive layers 23 of the electrode stack 203 is connected to the same connection pad 50 via the wiring 40.

The electrode stack 203 includes one sacrificial layer 21, one bent layer 22 stacked on the sacrificial layer 21, and the three conductive layers 23 stacked on the one bent layer 22. The three conductive layers 23 are stacked in a state of being in parallel. The wirings 40 connected to the three conductive layers 23 extend in the same direction.

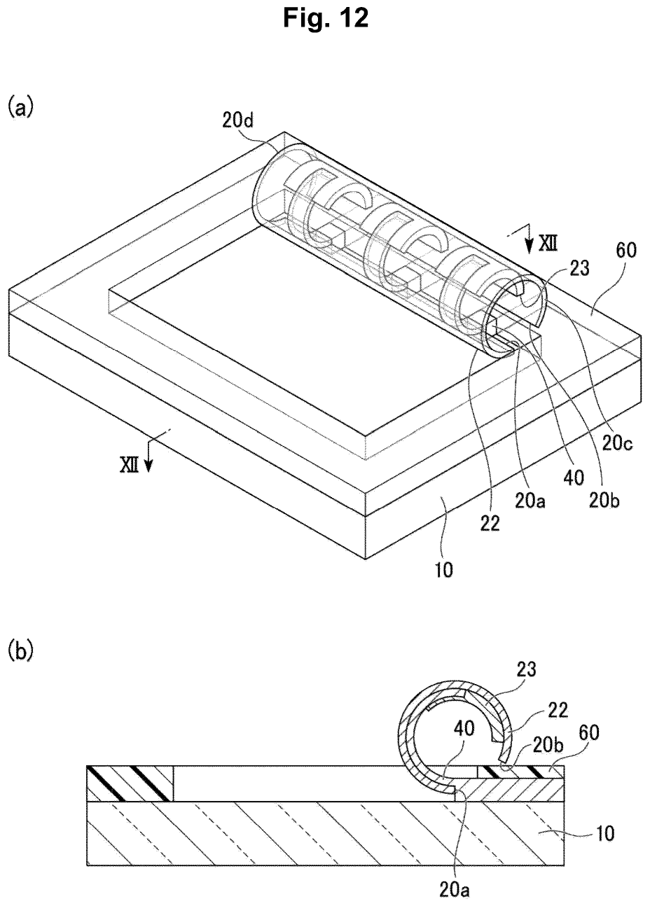

FIG. 12 is a diagram illustrating an example of a state in which the electrode illustrated in FIG. 11 is curved, FIG. 12(a) is a perspective diagram, and FIG. 12(b) is a cross-sectional diagram taken along line XII-XII of FIG. 12(a).

When the sacrificial layer 21 is removed in the electrode stack 203 illustrated in FIG. 11 and the planar substrate 10 and the bent layer 22 are separated, as illustrated in FIG. 12, the bent layer 22 is spontaneously curved and forms the curved electrode 30 having a cylindrical shape. The resulting curved electrode 30 having a cylindrical shape is curved such that the side portion 20a connected to the wiring 40 and the side portion 20b opposite to the side portion 20a are in contact with each other.

With the electrode stack 203, spatiotemporal measurement is performed with respect to the cell population encapsulated when the curved electrode 30 is formed. For example, when the curved electrode 30 has a cylindrical shape (tubular shape), the electrical activity of the cells propagating from one end portion to the other end portion of the cylinder can be visualized, and the propagation characteristic of the electrical activity can be quantified. In addition, with the electrode stack 203, the shapes of the conductive layer 23 and the curved electrode 30 can be set independently of each other. In general, the size of the conductive layer 23 affects the electrode characteristics, and the optimum size for measurement may be different from the size of the curved electrode 30. For example, in a case where the size of the curved electrode 30 is as large as 1000 μm×1000 μm, when the size of the conductive layer 23 is also 1000×1 mm, the amplitude of the extracellular potential may decrease, and, in this case, the amplitude of the extracellular potential can be increased by setting the size of the conductive layer 23 to, for example, 50 μm×50 μm.

### [Curved Electrode Array]

FIG. 12 is a perspective diagram illustrating an example of a curved electrode array according to an embodiment of the present invention.

A curved electrode array 3 illustrated in FIG. 12 includes the planar substrate 10, a plurality of curved electrodes 30 disposed on the planar substrate 10, and a curved reference electrode 31. The curved electrode 30 is obtained by curving the bent layer 22 and the conductive layer 23 of the electrode stack 20 described above. The curved reference electrode 31 is obtained by curving the bent layer 22 and the conductive layer 23 of the reference electrode stack 26 described above. Each of the curved electrode 30 and the curved reference electrode 31 includes a bent layer curved to form an internal space and a conductive layer disposed on an inner surface of the bent layer.

### [Method for Manufacturing Curved Electrode Array]

A method for manufacturing the curved electrode array 3 of the present embodiment will be described by taking a case where the above-described electrode stack array 1 is used as an example.

The curved electrode array 3 of the present embodiment can be manufactured by removing the sacrificial layer 21 of the electrode stack array 1, separating the planar substrate 10 and the bent layer 22, and curving the bent layer 22 so that the surface on a side opposite to the surface on the sacrificial layer 21 side becomes an inner side to form the curved electrode 30. The method for removing the sacrificial layer 21 varies depending on the material of the sacrificial layer 21. For example, when the material of the sacrificial layer 21 is a water-soluble inorganic material or a water-soluble polymer material, a method for bringing water into contact with the sacrificial layer 21 can be used. When the material of the sacrificial layer 21 is calcium alginate gel, a method of bringing a chelating agent such as sodium citrate or ethylenediaminetetraacetic acid (EDTA), or an enzyme such as alginate lyase into contact with the sacrificial layer 21 can be used.

The curved electrode array 3 of the present embodiment configured as described above can measure the extracellular potential at a higher S/N using the microelectrode array measurement method by encapsulating the cells in the curved electrode 30.

### [Method for Measuring Extracellular Potential]

A method for measuring the extracellular potential of the present embodiment will be described by taking a case where the above-described electrode stack array 1 is used as an example. In the method for measuring the extracellular potential of the present embodiment, the extracellular potential is measured by steps (1) to (3) described below.

(1) A step of attaching the cells to the surface of the conductive layer 23 of the electrode stack array 1. As a method for attaching the cells to the surface of the conductive layer 23, for example, a method for injecting a cell-containing culture solution to the inside of the culture ring 70 and bringing the cell-containing culture solution into contact with the conductive layer 23 can be used. In this step, it is preferable that the cell-containing culture solution is not brought into contact with the conductive layer 23 of the reference electrode stack 26.

(2) A step of removing the sacrificial layer 21 of the electrode stack array 1, detaching the planar substrate 10 and the bent layer 22 to curve the bent layer 22 so that the surface on a side opposite to the surface on the sacrificial layer 21 side becomes an inner side so as to form the curved electrode array 3. This step is the same as the method for manufacturing the curved electrode array described above.

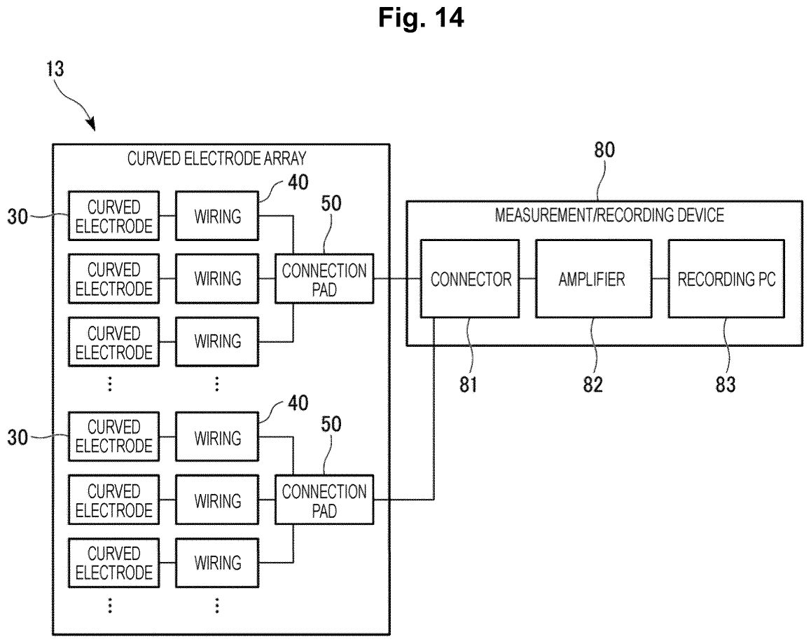

(3) A step of measuring the extracellular potential of the cells attached to the conductive layer 23 of the curved electrode 30 of the curved electrode array 3. FIG. 14 is a block diagram of a measurement device that can be used for carrying out the method for measuring an extracellular potential according to an embodiment of the present invention.

The extracellular potential of the cells can be measured using the measurement device illustrated in FIG. 14. A measurement device 80 illustrated in FIG. 14 includes a connector 81, an amplifier 82, and a recording personal computer (PC) 83. The connector 81 has a multi-channel probe and is connected to the connection pad 50 of the curved electrode array 3. The amplifier 82 includes amplification equipment that amplifies an electric signal and a bandpass filter that extracts a signal of a specific frequency band from the amplified signal. The recording PC 83 includes a recording unit that performs A/D conversion on a signal and records the converted signal as a digital signal.

The extracellular potential of the cells is measured using the measurement device 80 in the manner described below.

An electric signal detected by the conductive layer 23 of the curved electrode 30 is sent to the connector 81 via the wiring 40 and the connection pad. The electric signal sent to the connector 81 is amplified by the amplifier 82, and the extracellular potential in a specific frequency band is extracted by band pass processing. The extracted extracellular potential is A/D converted by the recording PC 83 and recorded as a digital signal.

In the method for measuring an extracellular potential of the present embodiment configured as described above, the cells are attached to the planar conductive layer 23 of the electrode stack 20, and the extracellular potential is measured in a state where the cells are encapsulated in the curved electrode 30, so that the extracellular potential can be measured with a higher S/N.

Although the embodiment of the present invention has been described with reference to the drawings so far, the specific configuration of the present invention is not limited to this embodiment, and includes a design and the like without departing from the gist of the present invention.

### EXAMPLES

### First Example: Production of Electrode Stack Array

As a substrate, an alkali-free glass substrate having a length of 50 mm, a width of 50 mm, and a thickness of 750 μm was prepared. A sodium alginate solution was applied to the surface of the alkali-free glass substrate by a spin coating method so as to have a film thickness of 200 nm, and the obtained applied layer was immersed in a calcium chloride solution to form a calcium alginate gel layer (sacrificial layer). Then, a single-layer graphene layer was transferred onto each sacrificial layer, and a poly-para-xylene layer was vapor-deposited thereon to form a bent layer including a two-layer stack. The single-layer graphene was an atomic layer, and by setting the film thickness of the poly-para-xylene layer to 100 nm, a bent layer of 100 nm was obtained.

Next, gold wiring having a thickness of 100 nm was provided by a sputtering method and a wet etching method. The wiring was extended to a part of the upper surface of each bent layer so that one end portion was connected to a next conductive layer to be formed.

Next, the single-layer graphene was transferred onto the bent layer to form a conductive layer. The stack including the sacrificial layer, the bent layer, and the conductive layer was processed using ionic etching with oxygen plasma through a mask including a photoresist to form a plurality of electrode stacks and a reference electrode stack having a size of 200 μm×400 μmm. A shaft portion was provided in the conductive layer of the electrode stacks and the reference electrode stack. In addition, in order to promote the exchangeability of the culture solution, a small hole having a diameter of 6 μm was formed in the surface of the conductive layer.

A poly-para-xylene layer (insulating layer) was formed by a vapor phase growth method with a mask left on the surface of the conductive layer of the electrode stack. Moreover, a mask was formed on the insulating layer again, and the insulating layer was removed only around the electrode stack and the connection pad using ionic etching with oxygen plasma. The mask after use was removed with acetone. Finally, a borosilicate glass ring having an inner diameter of 20 mm was adhered using a polydimethylsiloxane adhesive so as to surround the conductive layers of the plurality of electrode stacks to form a culture ring. In this way, an electrode stack array was produced.

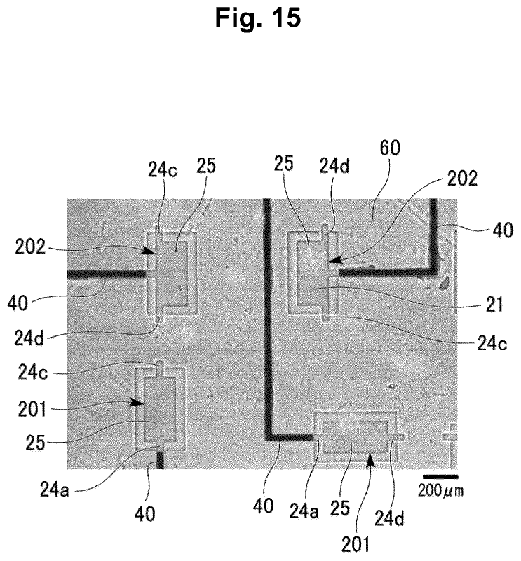

An optical microscope image of an electrode portion of the obtained electrode stack array is illustrated in FIG. 15. FIG. 15 illustrates the opening type electrode stack 201 in which the wiring 40 is connected to the opening of the curved electrode when the electrode is curved in a cylindrical shape and the side surface portion type electrode stack 202 in which the wiring 40 is connected to the side surface portion of the curved electrode when the electrode is curved in a cylindrical shape. The opening type electrode stack 201 includes the shaft portion 24a and the shaft portion 24b. The side surface portion type electrode stack 202 includes the shaft portion 24c and the shaft portion 24d.

### Second Example: Encapsulation of Cells

A cardiomyocyte-containing culture solution collected from a rat fetus was dropped onto the conductive layer of the electrode stack of the electrode stack array produced in the first example. Next, EDTA was rapidly injected into the cardiomyocyte-containing culture solution dropped on the conductive layer to dissolve the sacrificial layer (calcium alginate gel layer) of the electrode stack. When the sacrificial layer was dissolved, the bent layer was curved in a cylindrical shape, and a curved electrode array having a curved electrode having a cylindrical shape encapsulating cardiomyocytes was formed. Thereafter, the cardiomyocyte-containing culture solution that was not encapsulated in the curved array was removed, only the culture solution was injected into the culture ring of the electrode stack array, and EDTA was injected into the injection solution to dissolve the sacrificial layer of the reference electrode stack. Thus, a curved reference electrode was formed. The cardiomyocytes were cultured for five days using the obtained curved electrode array. On Day 5 of culture, a tissue by a cell population was formed in the curved electrode having a cylindrical shape. FIG. 5 illustrates the appearance of the encapsulated cardiomyocytes as a differential interference microscope image and a stained image with an Anti-Crdiac troponin T antibody. The cell population was engrafted along the curved electrode having a cylindrical shape, and it was confirmed that patterning of cells was possible by the present method.

### Third Example: Increase in Measurement Signal due to Bending of Electrode

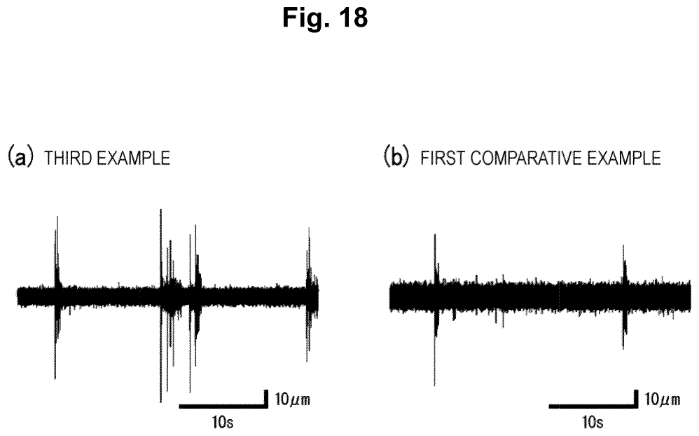

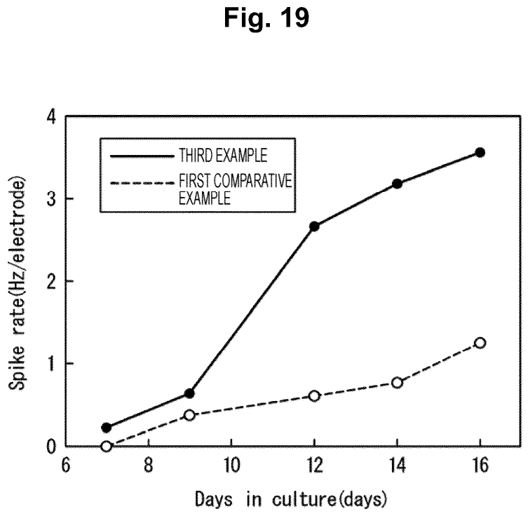

A curved electrode array having a curved electrode having a cylindrical shape encapsulating nerve cells was obtained in the same manner as in the second example except that nerve cells collected from a rat fetus were used instead of cardiomyocytes collected from a rat fetus. The nerve cells were cultured for 16 days using the obtained curved electrode array. During culture of the nerve cells, the extracellular potential was measured and recorded. The extracellular potential was measured and recorded using an MED64 system manufactured by Alpha MED. FIG. 18 illustrates a typical waveform of the extracellular potential on Day 16 of culture. In addition, spike rate on each culture day was measured by the method described below. The results are illustrated in FIG. 19.

(Method for Measuring Spike Rate)

A negative peak exceeding a threshold value was regarded as a spike, the threshold value being five times the standard deviation at the extracellular potential. The number of spikes of each electrode was counted and converted into the number of spikes per second to calculate a spike rate, and the average value of all electrodes for which spikes were detected was used as a representative value.

### First Comparative Example

The nerve cells were cultured for 16 days and the extracellular potential was measured in the same manner as in the third example except that the nerve cells were cultured in the state of the electrode stack array, that is, a nerve cell-containing culture solution was dropped, and then the sacrificial layer was dissolved, and the curved electrode was not formed. FIG. 18 illustrates a typical waveform of the extracellular potential on Day 16 of culture. In addition, FIG. 19 illustrates the spike rate measurement results on each culture day.

From the results of FIG. 18, when comparing the third example in which nerve cells were cultured using the curved electrode with the first comparative example in which nerve cells were cultured using a planar electrode, it is found that the extracellular potential obtained in the third example shows more variations in potential indicating a typical spontaneous activity of nerve cells. In addition, from the results of FIG. 19, it can be seen that in the third example, the spike rate indicating spontaneous activity of nerve cells is also larger than that in the first comparative example, and in particular, the spike rate remarkably increases when the number of days of culture exceeds nine days. These results are considered to be an effect of suppressing the diffusion of the growth factor of the nerve cells and the current flowing through the conductive layer by using the curved electrode.

### INDUSTRIAL APPLICABILITY

The present invention can be applied to a measurement device that measures an extracellular potential of various cells using the microelectrode array measurement method.

### REFERENCE SIGNS LIST

1, 2 electrode stack array
3 curved electrode array
4 cell
10 planar substrate
20 electrode stack
20b, 20c, 20d side portion
201 opening type electrode stack
202 side surface portion type electrode stack
21 sacrificial layer
22, 22a, 22b bent layer
23 conductive layer
24a, 24b, 24c, 24d shaft portion
25 pore
26 reference electrode stack
30 curved electrode
31 curved reference electrode
40 wiring
50 connection pad
60 insulating layer
70 culture ring
80 measurement device
81 connector
82 amplifier
83 recording PC

1. An electrode stack array comprising:
a planar substrate; and
a plurality of electrode stacks, each electrode stack including
a sacrificial layer that is stacked on the planar substrate,
a bent layer that is stacked on the sacrificial layer and is capable of bending such that a surface on a side opposite to a surface on a side of the sacrificial layer becomes an inner side, and
a conductive layer that is disposed on the bent layer.

2. The electrode stack array according to claim 1, wherein
the bent layer and the conductive layer are made of a material having optical transparency.

3. The electrode stack array according to claim 1 or 2, wherein
the conductive layer includes a conductive carbon material.

4. A curved electrode array comprising:
a planar substrate; and
a plurality of curved electrodes, each curved electrode including
a bent layer that is curved to form an internal space, and
a conductive layer that is disposed on an inner surface of the bent layer.

5. The curved electrode array according to claim 4, wherein
cells are present in the internal space.

6. The curved electrode array according to claim 4, wherein
a shape of the bent layer is a tubular shape.

7. A method for manufacturing a curved electrode array, comprising:
a step of preparing the electrode stack array according to claim 1; and
a step of removing the sacrificial layer of the electrode stack array and detaching the planar substrate and the bent layer so as to curve the bent layer such that a surface on a side opposite to a surface on a side of the sacrificial layer becomes an inner side.

8. A method for measuring an extracellular potential, comprising:
a step of preparing the electrode stack array according to claim 1;
a step of attaching a cell to a surface of the conductive layer of the electrode stack array;
a step of forming a curved electrode array by removing the sacrificial layer of the electrode stack array and detaching the planar substrate and the bent layer so as to curve the bent layer such that a surface on a side opposite to a surface on a side of the sacrificial layer becomes an inner side; and
a step of measuring an extracellular potential of the cell attached to the conductive layer of a curved electrode of the curved electrode array.
